(19) United States
(12) Patent Application Publication
(10) Pub. No.: US 2024/0316109 A1
(43) Pub. Date: Sep. 26, 2024

(54) PHARMACEUTICAL COMPOSITION FOR TREATING BONE DISEASES

(71) Applicant: PUREC CO., LTD., Izumo-shi, Shimane (JP)

(72) Inventors: Takeshi TAKETANI, Shimane (JP); Yasuaki ODA, Shimane (JP); Yumi IYOKU, Shimane (JP); Takashi SUYAMA, Shimane (JP)

(73) Assignee: PUREC CO., LTD., Izumo-shi, Shimane (JP)

(21) Appl. No.: 18/277,618

(22) PCT Filed: Feb. 18, 2022
(86) PCT No.: PCT/JP2022/008103

§ 371 Date: Aug. 17, 2023

(30) Foreign Application Priority Data
Feb. 19, 2021 (JP) 2021-024794

(51) Int. Cl. A61K 35/28; A61K 35/51; A61P 19/08

(52) U.S. Cl. CPC A61K 35/28; A61K 35/51; A61P 19/08

(57) ABSTRACT

The present invention provides a pharmaceutical composition for increasing osteoblasts in a subject, which comprises high-purity mesenchymal stem cells, wherein the pharmaceutical composition is used in combination with hematopoietic stem cell transplantation to the subject.

### TECHNICAL FIELD

The present invention relates to a pharmaceutical composition for treating bone diseases.

### BACKGROUND ART

Hypophosphatasia (HPP) is an autosomal recessive inherited disease caused by mutations in the ALPL gene, and leads to a mineralization defect due to lack of alkaline phosphatase (ALP). Moreover, pyrophosphate accumulated with decrease in ALP activity disturbs mineralization, and the local concentration of phosphorus is reduced, which in turn causes hypomineralization and rickets-like changes in bones.

In serological aspects, substrates for ALP, i.e., phosphoethanolamine, inorganic pyrophosphate and pyridoxal 5′-phosphate are found to be elevated. As bone-related clinical features, bone curvature, bone fragility, tooth loss and so on are confirmed, while convulsion, hypoacusis and developmental retardation are confirmed as central nerve symptoms. Severe cases may follow a lethal clinical course, and even in mild cases, activities of daily living are often limited. Particularly in patients with respiratory disturbance due to hypoplasia of the thoracic cage including ribs, the vital prognosis is poor. The mortality of most severe patients who experience onset during the perinatal period is 50% to 100%. The number of patients is now estimated to be 100 to 200 in Japan, and no effective therapy has been established (only symptomatic therapies are available).

Currently available therapy for HPP is an ALP enzyme replacement therapy. Upon administration of this enzyme formulation, bone mineralization is improved not only to the amelioration of the vital prognosis of severe HPP patients, but also to allow recovery of the motor function even in mild cases. However, there are problems in the following aspects: since this therapy is a replacement therapy, the enzyme formulation is required to be administered continuously throughout the patient's lifetime (injected subcutaneously three times a week); antibodies against the enzyme are produced to attenuate the effect of the therapy; the enzyme formulation does not cross the blood-brain barrier and thus does not improve symptoms of the central nervous system; and high medical costs are required for this therapy (20 million yen or more per year is required even in the case of a patient taking the lowest dose), etc.

On the other hand, mesenchymal stem cells (MSCs) can be relatively easily separated from various tissues such as bone marrow, fat tissue, placenta, dental pulp, amniotic membrane, etc. With a focus on their tissue repair action and their immunosuppression ability, several hundreds of clinical studies on many diseases are currently in progress. However, only a few studies have entered the late clinical stage, and medicines approved by regulatory authorities in individual countries are limited in number, including Temcell (JCR-Pharma) for severe GvHD in Japan, and TiGenix's Alofisel (for anal fissure due to Crohn's disease) which was initially recommended for approval in Europe in 2019, etc.

As to the causes of such difficulties in the practical use of MSCs, various causes have been pointed out, including difficulty in quality control when cells to be administered are prepared uniformly in large quantity, difficulty in delivery of the administered cells to the affected area, and lack of non-invasive cell tracking techniques (i.e., difficulty in understanding in vivo kinetics), etc.

In previous studies, the inventors of the present invention have elucidated that when using two antibodies against LNGFR (CD271) and Thy1 (CD90), respectively, human MSCs can be selected with extremely high purity and quality, and the inventors have established a method for direct separation of human MSCs from bone marrow, peripheral blood, placental chorion and dental pulp with a cell sorter. A group of cells (Rapidly Expanding Clone: REC) obtained by this method is excellent in proliferation capacity, differentiation capacity and migration ability, and has been confirmed to be high-quality and high-purity human MSCs (Patent Document 1, Non-patent Document 2).

Moreover, the inventors of the present invention have performed allogeneic bone marrow-derived MSC transplantation together with bone marrow transplantation on severe HPP patients under the “Guidelines on Clinical Research Using Human Stem Cells” (Non-patent Document 1). This clinical study suggested that when MSCs were transplanted after bone marrow transplantation for replacement with donor-derived immunocytes (bone marrow cells), some of the transplanted MSCs were engrafted, without being rejected, into bone marrow to form normal bone.

### PRIOR ART DOCUMENTS

### Patent Documents

Patent Document 1: Japanese Patent No. 6363950
Patent Document 2: WO2016/017795

### Non-Patent Documents

Non-patent Document 1: T. Taketani et al., “Ex vivo expanded allogeneic mesenchymal stem cells with bone marrow transplantation improved osteogenesis in infants with severe hypophosphatasia,” Cell Transplant., vol. 24, no. 10, pp. 19 31-1943, 2015
Non-patent Document 2: Mabuchi Y et al., “LNGFR Thy-1⁺ Vcam-1^{hi+} cells reveal functionally distinct subpopulations in mesenchymal stem cells,” Stem Cell Reports 1(2): 152-165, 2013

### SUMMARY OF THE INVENTION

### Problem to be Solved by the Invention

Clinically significant effects obtained upon allogeneic bone marrow-derived MSC transplantation in combination with bone marrow transplantation include (i) improvements in bone mineralization and vital prognosis, and (ii) recovery of symptoms in the central nervous system and mental development. However, to form normal bone structures in patients receiving MSC transplantation and thereby obtain further efficacy, it is required to use high-purity MSCs with higher migration ability and bone differentiation capacity.

### Means to Solve the Problem

As a result of extensive and intensive efforts made to solve the above problem, the inventors of the present invention have succeeded in solving the above problem by conducting the administration of high-purity mesenchymal stem cells in combination with hematopoietic stem cell transplantation, thus leading to the completion of the present invention.

Namely, the present invention is as shown below.

[1] A pharmaceutical composition for increasing osteoblasts in a subject, which comprises high-purity mesenchymal stem cells, wherein the pharmaceutical composition is used in combination with hematopoietic stem cell transplantation to the subject.
[2] The pharmaceutical composition according to [1] above, wherein the hematopoietic stem cells are derived from umbilical cord blood.
[3] The pharmaceutical composition according to [1] or [2] above, wherein the hematopoietic stem cells are derived from the bone marrow of a donor other than the subject.
[4] The pharmaceutical composition according to any one of [1] to [3] above, wherein the subject is a patient with a congenital skeletal disease.
[5] The pharmaceutical composition according to [4] above, wherein the congenital skeletal disease is hypophosphatasia.
[6] The pharmaceutical composition according to any one of [1] to [5] above, wherein the high-purity mesenchymal stem cells are human bone marrow-derived rapidly proliferating mesenchymal stem cells.
[7] The pharmaceutical composition according to [6] above, wherein the rapidly proliferating mesenchymal stem cells are a cell population of stem cell clones co-positive for LNGFR (CD271) and Thy-1 (CD90), and meet at least one of the following features (a) and (b):
(a) the coefficient of variation for forward scatter in flow cytometry is 40% or less; and
(b) the average size of the cells is 20 μm or less.
   [7-1] The composition according to [6] above, wherein the human bone marrow-derived high-purity mesenchymal stem cells are a cell population of rapidly proliferating mesenchymal stem cell clones separated on the basis of being positive for LNGFR (CD271) or co-positive for LNGFR (CD271) and Thy-1 (CD90), and meet at least one of the following features (a) and (b):
(a) the coefficient of variation for forward scatter in flow cytometry is 40% or less; and
(b) the average size of the cells is 20 μm or less.
   [7-2] The composition according to [6] above, wherein the human bone marrow-derived high-purity mesenchymal stem cells are a cell population of rapidly proliferating mesenchymal stem cell clones derived from cells positive for LNGFR (CD271) or co-positive for LNGFR (CD271) and Thy-1 (CD90), and meet at least one of the following features (a) and (b):
(a) the coefficient of variation for forward scatter in flow cytometry is 40% or less; and
(b) the average size of the cells is 20 m or less.
   [8] The pharmaceutical composition according to any one of [1] to [7-2] above, wherein the cells are administered at the dose of 1×10⁷ cells per kg body weight of the subject and this administration is repeated weekly four times.
   [9] The pharmaceutical composition according to [8] above, wherein the cells are used at a concentration of at least 1×10⁶ cells/ml.
   [10] The composition according to any one of [1] to [9] above, wherein the HLA of the donor of the high-purity mesenchymal stem cells does not match the HLA of the subject.
   [11] The composition according to any one of [1] to [9] above, wherein the HLA of the donor of the high-purity mesenchymal stem cells matches at least 4 antigens among 6 antigens at three loci in the HLA of the subject.
   [12] The composition according to any one of [1] to [9] above, wherein the HLA of the donor of the high-purity mesenchymal stem cells matches at least 3 antigens among 6 antigens at three loci in the HLA of the subject.

### Effects of the Invention

The present invention enables the increase of osteoblasts in a subject. As a result, the pharmaceutical composition of the present invention can be used against congenital skeletal disease such as hypophosphatasia.

### BRIEF DESCRIPTION OF THE DRAWINGS

FIG. 1 shows transplantation test procedures to verify the efficacy of high-purity mesenchymal stem cell REC-01 on hypophosphatasia model mice.

FIG. 2 shows the survival curve of Alp1^{-/-} mice upon administration of asfotase alfa (Strensiq®) (the results verified for life-prolonging effect).

FIG. 3 shows the time course of changes in the body weight of each mouse after cell transplantation. The figure shows body weight changes over time in Alp1^{-/-} mice (left panel) and Alp1^{+/-} mice (normal group: right panel) after cell transplantation. Alp1^{+/-} mice used as a control group are littermates of Alp1^{-/-}. The vertical axis represents the time course of changes in body weight after transplantation relative to the body weight on the day of transplantation which was set to 1. The horizontal axis represents days after transplantation.

FIG. 4 shows the results examined for ALP activity in bone marrow at 2 months after REC-01 transplantation. The figure shows ALP staining images of frozen sections of femurs from Alp1^{-/-} mice administered with mouse bone marrow cells alone (left panel), Alp1^{-/-} mice administered with mouse bone marrow cells+REC01 (center-left and center-right panels), and Alp1^{-/-} mice serving as a positive control of ALP activity (right panel). Arrows (blue) indicate sites positive for ALP staining (bluish purple).

FIG. 5 shows long-term engraftment and ALP activity in bone marrow upon REC-01 transplantation. The figure shows ALP staining images of frozen sections of mouse femur and skull at 3 months after transplantation. Arrows (blue) indicate sites positive for ALP staining (bluish purple).

FIG. 6 shows the survival curve of Alp1^{-/-} mice in the presence or absence of REC-01 transplantation.

### DESCRIPTION OF EMBODIMENTS

### 1. Summary

The mechanism by which cell-based therapy exerts a therapeutic effect is generally divided into the mechanism based on cellular replacement, in which the transplanted cells are incorporated into the recipient's tissue and organ to replace the recipient's cells with the transplanted cells, thereby achieving functional recovery, and the mechanism expected to provide trophic action, in which the protection effect on host cells or the tissue repair ability is enhanced by the action of trophic factors, cytokines and extracellular matrixes produced from the transplanted cells, thereby achieving functional repair.

In the treatment of hypophosphatasia (HPP), cellular replacement is essential; and hence the distribution cells at the affected area, and their long-term survival and differentiation are the most important factors. Moreover, this disease requires intravenous systemic administration because it is a disease of osteogenesis imperfecta with systemic symptoms. However, commonly used mesenchymal stem cells (MSCs) mostly lose their migration ability, and have the risk of causing adverse events such as pulmonary embolism and clot formation when administered in abundance via the intravenous route, so that there is a limit in the number of cells that can be transplanted at one time. Moreover, in addition to HPP, there are 450 or more types of inherited osteogenesis diseases with no established therapy. Congenital skeletal diseases (CSDs) have no established therapy, and follow a lethal course or significantly disturb activities of daily living. In CSDs, damage occurs in the pathway of ossification from MSCs, which are the origin of bone-forming osteoblasts; and hence bone regenerative medicine with allogeneic MSCs whose ossification capacity is normal is considered to be a promising therapy.

The present invention has been completed with a focus on mesenchymal stem cells, particularly mesenchymal stem cells excellent in differentiation capacity and migration ability and with rapid proliferation capacity, i.e., high-purity mesenchymal stem cells. The present invention is characterized by being used to increase osteoblasts, and thus used as a medicament for bone diseases.

Namely, the present invention provides a pharmaceutical composition for increasing osteoblasts in a subject, which comprises high-purity mesenchymal stem cells. Moreover, the pharmaceutical composition of the present invention is used in combination with hematopoietic stem cell transplantation to a subject.

### 2. High-Purity Mesenchymal Stem Cells

Mesenchymal stem cells for use in the present invention are somatic stem cells derived from the mesodermal tissue (mesenchyme), and are expected for application to regenerative medicine, such as reconstruction of bones, blood vessels and myocardia.

Mesenchymal stem cells can be obtained from various tissues, such as bone marrow, fat tissue, placental tissue, dental pulp or umbilical cord tissue, etc. Their purification process is as shown below, by way of example.

A mixed population of cell types obtained by enzymatic treatment of a small amount of fat pieces taken from a human or a non-human mammal (e.g., cow, monkey, cat, mouse, rat, guinea pig, hamster, pig, dog, rabbit, sheep, horse and goat) is centrifuged to separate a population of floating fat cells. This population is allowed to stand in contact with the ceiling surface of a culture vessel filled with a culture solution, and fibroblast-like cells settling to the bottom surface and growing thereon are subcultured for proliferation.

Alternatively, in the present invention, it is also possible to use iPS cell-derived mesenchymal stem cells or commercially available mesenchymal stem cells.

In the previous studies, the inventors of the present invention have succeeded in isolating a rapidly proliferating cell clone (Rapidly Expanding Clone: REC) from among mesenchymal stem cells positive for LNGFR (CD271) (CD271⁺ cells) or mesenchymal stem cells co-positive for LNGFR (CD271) and Thy-1 (CD90) (CD271⁺ CD90⁺ cells). This REC clone is a kind of “high-purity mesenchymal stem cells” in the present invention.

REC is a cell line capable of reaching confluence within 2 weeks when single-cell sorted into 96-well plate and cultured and has proliferation capacity, differentiation capacity and migration ability which are all 1000-fold or more higher than those of mesenchymal stem cells obtained in a conventional manner. Moreover, particularly because of retaining migration ability, REC can be administered via the intravenous route, and therefore can be applied for use in serious systemic diseases such as osteochondrodysplasia.

REC, a kind of high-purity mesenchymal stem cells, is a very uniform cell population, because cells positive for LNGFR (CD271) or co-positive for LNGFR (CD271) and Thy1 (CD90) are separated with a cell sorter from the mononuclear cell fraction of bone marrow, and single-cell seeded into lates to select only rapidly proliferating MSCs with high proliferation potency.

REC thus separated and purified is capable of multiplying from a single cell to 1012 cells or more while retaining differentiation capacity, proliferation capacity and migration ability. Particularly because of retaining migration ability, REC can be administered via the intravenous route, and therefore can be expected for use in serious systemic diseases such as osteochondrodysplasia.

In the present invention, it is possible to use cell clones with smaller variations in their differentiation capacity and proliferation capacity among the above REC clones.

In the present invention, mesenchymal stem cells positive for LNGFR (CD271) or co-positive for LNGFR (CD271) and Thy-1 (CD90) may be obtained, for example, in accordance with the method described in WO2009/31678. The outline of this method is as follows.

First, a cell population containing human mesenchymal stem cells is sorted to select a cell fraction positive for LNGFR (CD271) (CD271⁺) or co-positive for CD271 and CD90 (CD271⁺ CD90⁺), thereby highly enriching mesenchymal stem cells. It should be noted that when a cell population containing human mesenchymal stem cells is contaminated with blood cells, the step of sorting for cells co-negative for CD45 and CD235a (CD45⁻ CD235a⁻) may be added so as to separate non-blood cells.

A cell population containing mesenchymal stem cells may be prepared by flow cytometry or affinity chromatography.

Any material may be used to obtain this cell population, and examples include bone marrow, fat tissue, umbilical cord blood, peripheral blood (including peripheral blood after G-CSF administration), etc. It should be note that bone marrow used for this purpose may be vertebral, sternal or iliac bone marrow, etc. In addition, the cells may be exemplified by ES cells and iPS cells.

If the material forms cell aggregates incorporating mesenchymal stem cells during cell preparation, the material may optionally be subjected to physical treatment by pipetting, etc., or enzymatic treatment with trypsin, collagenase, etc. In addition, if the material is contaminated with red blood cells, it is preferable to hemolyze the red blood cells in advance.

Using the cell population prepared as above, CD271⁺ cells or CD271⁺ CD90⁺ cells are selected.

To select CD271⁺ cells or CD271⁺ CD90⁺ cells, antibody-based techniques may be used, by way of example. The antibody used for this purpose is an anti-CD271 antibody and/or an anti-CD90 antibody capable of selecting CD271⁺ cells or CD271⁺ CD90⁺ cells. When flow cytometry is used for selection, an anti-CD271 antibody or anti-CD271 and anti-CD90 antibodies, which are labeled with different fluorescent dyes (e.g., FITC, PE, APC), are used in combination as appropriate, whereby living cells can be selected within a short period of time. In addition to flow cytometry, magnetic bead-based techniques or affinity chromatography-based techniques can also be used to select CD271⁺ CD90⁺ cells.

It should be noted that before using these techniques, dead cells may be removed in advance by reacting the cell population with a fluorescent dye for staining dead cells (e.g., PI) to remove fluorescently stained cells.

Then, the selected LNGFR-positive cells or LNGFR and Thy1 co-positive cells are subjected to single cell (clone) culture to select rapidly expanding lots, thereby obtaining RECs excellent in proliferation capacity, differentiation capacity and migration ability.

As used herein, the term “rapidly expanding” or “rapidly proliferating” is intended to mean that when single-cell seeded and cultured into each well of a 96-well culture plate, cells have a proliferation rate such that the culture plate is confluent or semi-confluent at 2 weeks or earlier after the initiation of culture (i.e., have a doubling time of 26±1 hours).

The term “confluence” or “confluent” refers to a state where 90% or more of the culture vessel surface (culture surface) is coated with cultured cells. Likewise, the term “semi-confluence” or “semi-confluent” refers to a state where 70% to 90% of the culture vessel surface (culture surface) is coated with cultured cells. The size and type of culture devices to be used may be changed as appropriate depending on the growth rate of cells. Cells whose expansion is delayed (Moderately/Slowly Expanding Cells), i.e., cells which have not reached semi-confluence or confluence even at 2 weeks after single cell culture are discarded. RECs collected from the wells selected as RECs are transferred to culture flasks on a well-by-well basis, and further cultured to reach confluence (expansion culture). Then, the cells after expansion culture are collected separately. REC from one well is defined as one lot.

REC for use in the present invention is obtained by single-cell sorting and single-cell seeding; and hence genetic traits are all the same among the cells grown. Thus, in the present invention, the term “clone” is used to refer to not only a cell population as a whole, but also individual cells constituting the cell population.

Moreover, in the present invention, RECs used for selection may be evaluated in advance with a REC marker (anti-Ror2). For example, after the above expansion culture, the cells grown in adherent state are collected from all lots, and an aliquot (about 1 to 3×10⁵ cells) of each lot is sampled and single-stained with a monoclonal antibody against anti-Ror2. Procedures for single staining with a monoclonal antibody against anti-Ror2 are known (WO2016/17795). In brief, flow cytometry analysis with the REC marker is conducted to determine the ratio of the REC marker-positive cells in the collected cells. For determination of this ratio, mRNA expression of Ror2 may be quantified by quantitative PCR, or alternatively, this ratio may be determined manually under a microscope. If the above positive ratio is equal to or greater than a given value (e.g., 65%), such a lot (cell population) is determined to be acceptable and may be used for selection.

A cell population containing the cell clones of the present invention is a population of mesenchymal stem cell clones which are co-positive for LNGFR (CD271) and Thy-1 (CD90) and which are rapidly proliferating, and this cell population meets at least one of the following features (a) and (b):
(a) the coefficient of variation for forward scatter in flow cytometry is 40% or less; and
(b) the average size of the cells is 20 μm or less.

In one embodiment of the present invention, the human bone marrow-derived high-purity mesenchymal stem cells are a cell population of rapidly proliferating mesenchymal stem cell clones separated on the basis of being positive for LNGFR (CD271) or co-positive for LNGFR (CD271) and Thy-1 (CD90), and meet at least one of the following features (a) and (b):
(a) the coefficient of variation for forward scatter in flow cytometry is 40% or less; and
(b) the average size of the cells is 20 μm or less.

In another embodiment of the present invention, the human bone marrow-derived high-purity mesenchymal stem cells are a cell population of rapidly proliferating mesenchymal stem cell clones derived from cells positive for LNGFR (CD271) or co-positive for LNGFR (CD271) and Thy-1 (CD90), and meet at least one of the following features (a) and (b):
(a) the coefficient of variation for forward scatter in flow cytometry is 40% or less; and
(b) the average size of the cells is 20 μm or less.

In the present invention, REC clones in the individual lots are examined for their proliferation potency, fat differentiation potency, REC-specific marker expression levels and cell size uniformity to analyze their correlations with each other, whereby high-purity and uniform RECs with high cell performance can be selected.

In the present invention, the coefficient of variation (CV) for forward scatter and the average size of cells are used as indicators for selection.

Forward scatter is light scattered forward at a small angle relative to the axis of a laser beam. Forward scatter is composed of scattered light, diffracted light and refracted light generated from a laser beam on the cell surface, and gives information about the size of a sample.

The coefficient of variation (CV) is the standard deviation divided by the mean, and is used to relatively evaluate variations in data with different units or the relation between data and variations relative to the mean.

In the present invention, a cell population is selected such that the above CV value is 40% or less. A cell population whose CV value is 40% or less is a cell population composed of cells of uniform size. Preferably, the CV value is 35% or less, 30% or less, 25% or less, or 20% or less.

Likewise, the average size of cells in the cell population selected by the present invention is 20 μm or less, preferably 18 μm or less, and is in the range of 14 μm to 18 μm.

### 3. The Composition of the Present Invention

### (1) Subject

The composition of the present invention is applied to humans or non-human organisms such as birds and non-human mammals (e.g., cow, monkey, cat, mouse, rat, guinea pig, hamster, pig, dog, rabbit, sheep, and horse) and is used to cause osteoblast proliferation and thereby promote bone regeneration.

The diseases to be applied are skeletal diseases which require the increase of osteoblasts, regardless of whether they are congenital or acquired. Skeletal diseases are a generic name for diseases causing skeletal abnormalities by damage to skeleton-forming tissues (e.g., bone and cartilage). Moreover, congenital skeletal diseases are divided into two major categories, i.e., dysostosis and osteochondrodysplasia.

Examples of congenital skeletal diseases include thoracic insufficiency syndrome, hypophosphatasia, achondroplasia, hypochondroplasia, osteogenesis imperfecta, marble bone disease, multiple cartilaginous exostosis, enchondromatosis, type II collagenopathy, chondrodysplasia punctata, pseudoachondroplasia, Larsen's syndrome, fibrodysplasia ossificans progressiva, TRPV4-associated disorders, osteosclerotic diseases, Beals syndrome and so on. Particularly preferred in the present invention is hypophosphatasia which is among congenital skeletal diseases.

### (2) Cell Concentration and Dosage

The cells are used as a cell population at a concentration of at least 1×10⁶ cells/ml. For example, the concentration is 1×10⁶ cells/ml, 5×10⁶ cells/ml, 1×10⁷ cells/ml, etc., and may be set as appropriate depending on the intended purpose of use. The cells are administered at the dose of 1×10⁷ cells per kg body weight of the subject and this administration is repeated weekly once to four times (the administration period per course is 1 to 4 weeks), preferably repeated four times.

### (3) Composition

In the present invention, mesenchymal stem cells are administered in the form of a pharmaceutical composition. In one embodiment, such a composition comprises a pharmaceutically acceptable carrier and/or excipient. The mode of administration is injection such as intravenous injection, intravenous drip infusion, etc.

The terms “carrier” and “excipient” each refer to a composition conventionally used in the art to facilitate cell storage, administration and/or biological activity.

Examples of a carrier for use in the composition of the present invention include physiological saline, aqueous dextrose, lactose, Ringer's solution, buffer and so on. Likewise, examples of an excipient include starch, cellulose, glucose, lactose and so on.

The composition of the present invention comprising mesenchymal stem cells may be prepared as an appropriate liquid suspension, for example, in a buffer solution or a culture solution. When used for injection, the suspension may contain sodium carboxymethylcellulose, sorbitol, dextran, etc.

### 4. Combination with Hematopoietic Stem Cell Transplantation

In most cases, when mesenchymal stem cells (MSCs) are simply administered via the intravenous route, only a few cells are engrafted in bone marrow. For this reason, the present invention is configured to administer mesenchymal stem cells in combination with hematopoietic stem cell transplantation.

First, MSCs derived from a subject (patient) whose bone formation ability has been damaged are replaced with MSCs whose bone formation ability is normal. To this end, patient-derived MSCs are removed with an anticancer agent or by radiotherapy, etc. In this case, patient-derived hematopoietic stem cells are also removed; and hence the high-purity mesenchymal stem cells of the present invention are administered in combination with hematopoietic stem cell transplantation to maintain blood cells. The phrase “(in) combination with” is intended to mean that the administration of high-purity mesenchymal stem cells and hematopoietic stem cell transplantation are used in one course of treatment, and the timing of hematopoietic stem cell transplantation may be either before or after the administration of high-purity mesenchymal stem cells, or may be simultaneous with the administration of high-purity mesenchymal stem cells. The phrase “simultaneous with” includes an embodiment where high-purity mesenchymal stem cells and hematopoietic stem cells are mixed with the above carrier to prepare a suspension, which is then injected. However, in the present invention, it is preferred that high-purity mesenchymal stem cells are administered after hematopoietic stem cell transplantation.

The transplanted hematopoietic stem cells differentiate into blood cells while recognizing the simultaneously administered MSCs as self, so that there is very little possibility that the transplanted MSCs will be rejected by the recipient's immunocytes.

In the present invention, a donor serving as a source of high-purity mesenchymal stem cells, and a donor serving as a source of hematopoietic stem cells for use in hematopoietic stem cell transplantation are both persons (e.g., healthy persons) who are different from the subject patient. In addition, the donor of high-purity mesenchymal stem cells and the donor of hematopoietic stem cells may be the same or different. In hematopoietic stem cell transplantation, the match rate of four HLA loci, i.e., HLA-A, -B, -C and -DR loci (8 antigens) is considered to be important, but the HLA of the donor of high-purity mesenchymal stem cells does not match the HLA of the subject in most cases. However, the present invention can be applied even when the HLA of the subject does not completely match the HLA of the donor.

In other words, it is possible to use high-purity mesenchymal stem cells whose donor's HLA matches at least 3 antigens or at least 4 antigens among 6 antigens at three loci (HLA-A, -B and -DR-B) in the HLA of the subject.

### EXAMPLES

The present invention will be further described in more detail by way of the following examples, which are not intended to limit the scope of the present invention.

### Preparation Example

High-purity mesenchymal stem cells for use in the present invention were obtained as follows: bone marrow aspirate taken from a healthy donor was separated into mononuclear cells by density gradient centrifugation, and CD90 and CD271 antibodies were used to selectively separate only co-positive cells, which were then dissociated into single cells and further expanded until P4 to select a cell clone whose CV value was 40% or less.

The resulting cell clone is designated as “REC-01.”

### Test Example

### Verification of Life-Prolonging Effect on Alp1^{-/-} Mice Upon Strensiq Administration

179 pathological model mice of hypophosphatasia (i.e., alkaline phosphatase-deficient mice: ALP^{(-/-)} mice) were prepared and bred, and these mice were administered subcutaneously every other day with Strensiq (asfotase alfa).

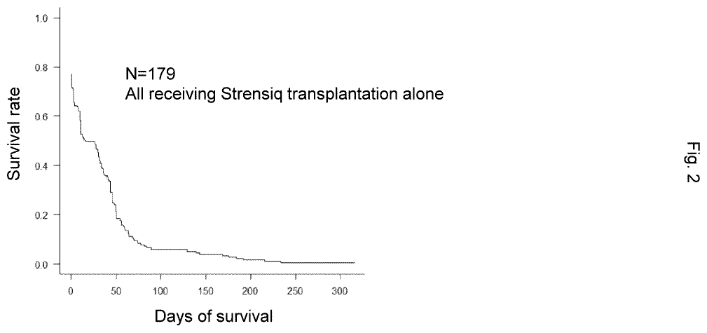

As a result, among the 179 Alp1^{-/-} mice, 41 mice (22.9%) died on the day of birth (including stillbirth cases). Mice survived after genetic typing were administered subcutaneously every other day with the enzyme (Strensiq). The results obtained are shown in FIG. 2. 88 mice (49.2%) died within 2 weeks after birth, 98 mice (54.7%) died within 30 days after birth, 158 mice (88.3%) died within 60 days after birth, and 168 mice (93.9%) died within 100 days after birth.

If Strensiq is not administered, all the mice will die within 2 weeks (Millan J L, et al. J Bone Miner Res. 2008). Thus, the administration of Strensiq alone gives a slight life-prolonging effect, but its effect on vital prognosis is deemed to be limited.

### Example 1

Transplantation Test of High-Purity Mesenchymal Stem Cell REC into ALP^{(-/-)} Mice

In this example, hypophosphatasia model mice, i.e., Alp1^{-/-} mice were used to evaluate the efficacy of REC-01 transplantation.

### 1. Method

### (1) REC-01 Administration to Recipient Mice

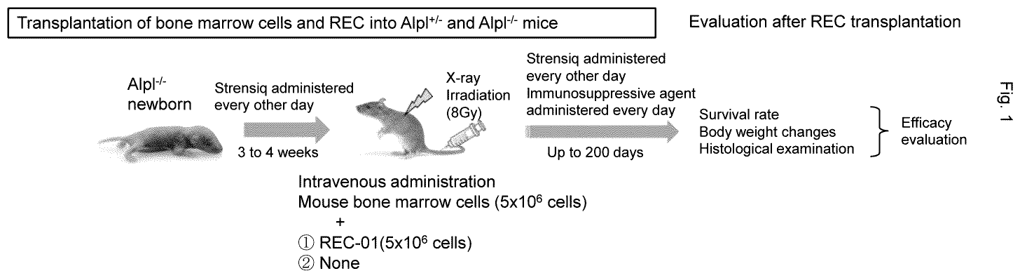

Genomic DNAs were extracted from the tails of pups on day 0 to day 5 obtained by crossing between Alp1 heterozygous (Alp1^{-/-)} mice, and then subjected to PCR for genotyping. After birth, Alp1^{-/-} mice were continuously administered subcutaneously with 8 mg/kg Strensiq at a frequency of twice or three times a week (FIG. 1).

On the day before REC-01 transplantation, wild-type mice (B6 female) serving as donors and Alp1^{-/-} mice serving as recipients were each administered with an immunosuppressive agent (0.5 mg/i mL Graceptor) in an amount of 0.1 mL per 10 g body weight. On the day of the transplantation, the Alp1^{-/-} mice at 33 to 159 days of age after birth (n=9) were fully sedated by being administered with a mixture of three anesthetic agents (0.75 mg/kg domitor, 4 mg/kg midazolam, 5 mg/kg vetorphale) in an amount of 0.1 mL per 10 g body weight. Then, the mice were irradiated with 8 Gy, and immediately thereafter 5×10⁶ cells of B6 mouse-derived bone marrow cells and 5×10⁶ cells of REC were suspended and administered through the mouse tail vein (liquid volume: 200 μL in total). Alp1^{-/-} mice in the control group were administered with the same number of bone marrow cells alone (FIG. 1).

(2) Maintenance after Cell Administration

The mice after cell transplantation were each administered almost every day with an immunosuppressive agent (0.5 mg/i mL Graceptor) in an amount of 0.1 mL per 10 g body weight. In addition, regardless of receiving or not receiving cell transplantation, the ALp1^{-/-} mice were continuously administered subcutaneously with 8 mg/kg Strensiq at a frequency of twice or three times a week until the Alp1^{-/-} mice died or were euthanized for experiments.

### (3) Survival Curve Analysis on Recipient Mice

The Kaplan-Meier survival curve was analyzed using the statistical analysis software EZR. For details of analysis procedures, reference was made to various existing manuals (Department of Hematology, Jichi Medical University Saitama Medical Center).

### 2. Results

(1) Time Course of Changes in the Body Weight of Each Mouse after Cell Transplantation

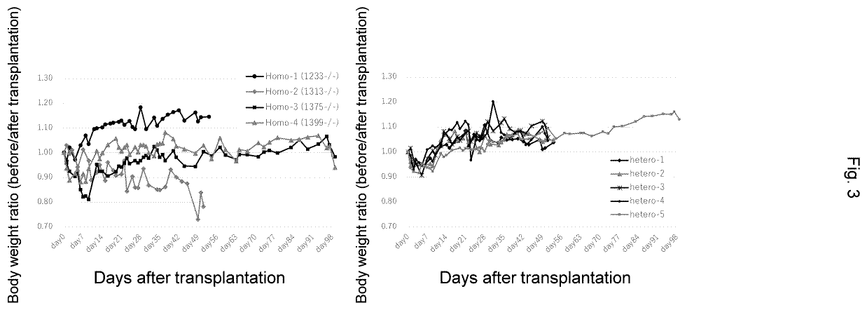

Alp1^{-/-} after REC-01 cell transplantation (n=4) and Alp^{-/-} serving as a normal group (n=5) were periodically measured for their body weight to observe changes over time in their body weight. The body weight of each mouse on the day of cell transplantation was set to 1, and the time course of changes in the body weight ratio after transplantation was shown on a graph (FIG. 3).

As a result, there were differences from recipient to recipient in the Alp1^{-/-} transplanted group. Homo-1 (#1233) was found to show body weight gain like the heterozygous mice, whereas Homo-2 (#1313) started to lose its body weight from one month after transplantation and died on day 50. On the other hand, Homo-3 (#1375) and Homo-4 (#1399) showed no great gain or loss in their body weight.

(2) Examination of ALP Activity in Bone Marrow after REC-01 Transplantation

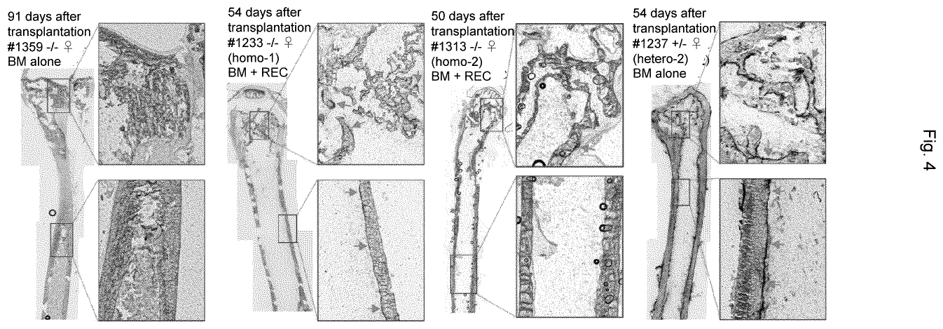

In the normal mouse (#1237, hetero-2) which was provided as a positive control of ALP activity, ALP activity (pale purple area) was detected in the femoral head spongy bone (near the growth plate) and the cortical bone surface, where many osteoblasts are usually localized (FIG. 4, right panel). In the mouse #1313 (homo-2; FIG. 4, center-right panel) whose body weight was reduced during the observation period after REC-01 transplantation, ALP activity was detected in only a few sites of the femur. Since ALP activity is a marker for osteoblasts, REC-01 is considered to have differentiated into osteoblasts. On the other hand, in the femur of #1359 transplanted with bone marrow cells (BM) alone (serving as a negative control), no ALP activity was detected in all sites (FIG. 4, left panel).

Noteworthily, among the Alp1^{-/-} mice receiving hematopoietic stem cell transplantation in combination with REC-01, #1233 (homo-1; FIG. 4, center-left panel) showing normal body weight gain showed the same result as the normal mouse, i.e., ALP activity was detected in the femoral head spongy bone and the cortical bone surface.

The ALP^{-/-} mice used as recipients were all continuously administered with Strensiq over the period from immediately after their birth until they died or were sacrificed as analytes. Since ALP activity was not detected at all in the group transplanted with bone marrow alone, it was suggested that Strensiq, which is an ALP formulation, when administered would be able to increase the blood ALP concentration, but would not remain in bone marrow, and that in animals showing body weight gain after REC-01 administration, REC-01 engrafted in bone marrow would differentiate into osteoblasts and thereby produce ALP, which in turn would contribute to body weight gain (growth).

Then, ALP staining was performed on the frozen sections of mouse femur and head at 100 days after transplantation, which is the technical time limit.

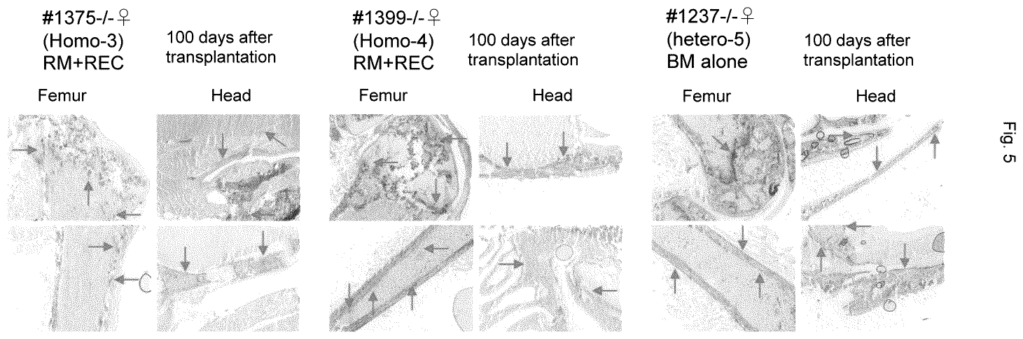

In the Alp1^{-/-} mice transplanted with BM+REC-01 (#1375 and #1399), ALP activity was detected in the epiphyseal region, the cortical bone and the spongy bone, as in the case of the normal mouse (#1237^{+/-}, hetero-5). Further, in their head sections, ALP activity, which was slightly weaker than in the femur sections, was detected in a few sites of the skull and in sites of probably other brain tissues (FIG. 5).

The survival period of mesenchymal stem cells in bone marrow has not been experimentally proven at all. However, in hematopoietic stem cell transplantation, if donor cells are detected even after 3 months have passed, it is defined as long-term engraftment (Osawa et al. Science 273 (5272): 242-5, 1996). When applying this definition, it can be determined that the long-term engraftment of REC-01 in bone marrow was confirmed.

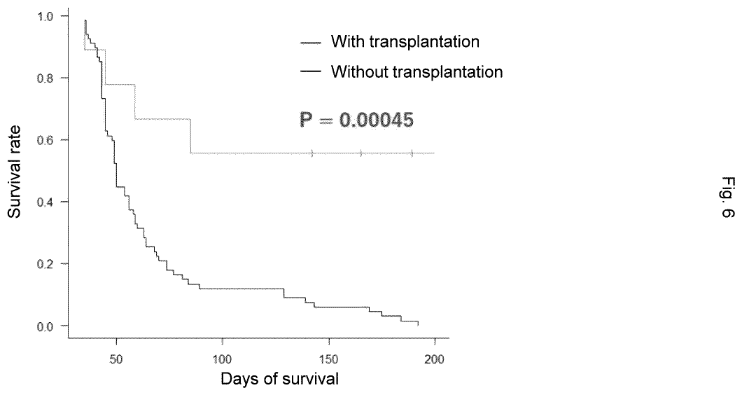

### (3) Improvement in Survival Rate in the Presence or Absence of REC-01 Transplantation (FIG. 6)

The survival curves of the non-transplanted group (n=67, with the enzyme, right panel: black line) and the transplanted group (n=9, BM+REC+with the enzyme, right panel: red line) were evaluated by the log-rank test using the Kaplan-Meier method.

As a result, REC-01 transplantation significantly improved the survival rate of Alp1^{-/-} mice (FIG. 6, p=0.00045).

The enzyme (Strensiq) does not cross the blood-brain barrier, and is therefore pointed out to have a problem in being unable to suppress epileptic seizure, which is one of the major symptoms of hypophosphatasia. In actual fact, in the group without transplantation (with the enzyme), mice which died of epileptic seizure were frequently observed.

In contrast to this, in the REC-01 transplanted group, epileptic seizure per se could hardly be confirmed, while ALP-positive cells were detected in the skull, as shown in FIG. 5. Thus, REC-01 administration was shown to be also effective in the suppression of epileptic seizure.

### 3. Conclusion

The Alp1^{-/-} recipient mice transplanted with REC-01 were confirmed to show normal ALP production in bone marrow (ALP activity confirmed by ALP staining) and the long-term engraftment of transplanted REC-01-derived human cells into the mouse femora (positive for ALP and STEM121 antibodies). Moreover, the REC-01 transplanted group was found to show a significant increase in the survival rate when compared to the non-transplanted group.

The foregoing results indicate that in addition to the clinical outcomes (increased survival rate) obtained by the administration of Strensiq alone, REC-01 transplantation can be expected to normalize the patient's bone tissue through the engraftment of transplanted REC-01 into the femur of a hypophosphatasia patient, its differentiation into osteoblasts, and normal bone-type ALP production therefrom, and further can be expected to provide a higher effect on the patient's QOL by functional improvements including the suppression of epileptic seizure, etc.

No cases have been reported, in either humans or mice, which show the successful long-term engraftment of cultured mesenchymal stem cells by intravenous administration.

In contrast to this, the present invention provides cross-species transplantation in combination with an immunosuppressive agent, where genetically modified animals whose immune system is normal are used as recipients, so that histocompatibility is extremely low. Thus, it is an amazing result that transplanted REC-01 was detectable over 3 months.

The present invention means success in the cross-species use of human mesenchymal stem cell transplantation in combination with hematopoietic stem cell transplantation by intravenous administration, and is very useful as a medicament for bone diseases.

1-14. (canceled)

15. A method for increasing osteoblasts in a subject, comprising:
administering to said subject a pharmaceutical composition which comprises high-purity mesenchymal stem cells, in combination with hematopoietic stem cell transplantation to the subject.

16. The method according to claim 15, wherein the hematopoietic stem cells are derived from umbilical cord blood.

17. The method according to claim 15, wherein the hematopoietic stem cells are derived from the bone marrow of a donor other than the subject.

18. The method according to claim 15, wherein the subject is a patient with a congenital skeletal disease.

19. The method according to claim 18, wherein the congenital skeletal disease is hypophosphatasia.

20. The method according to claim 15, wherein the high-purity mesenchymal stem cells are human bone marrow-derived rapidly proliferating mesenchymal stem cells.

21. The method according to claim 20, wherein the rapidly proliferating mesenchymal stem cells are a cell population of stem cell clones co-positive for LNGFR (CD271) and Thy-1 (CD90), and meet at least one of the following features (a) and (b):
(a) the coefficient of variation for forward scatter in flow cytometry is 40% or less; and
(b) the average size of the cells is 20 μm or less.

22. The method according to claim 20, wherein the human bone marrow-derived high-purity mesenchymal stem cells are a cell population of rapidly proliferating mesenchymal stem cell clones separated on the basis of being positive for LNGFR (CD271) or co-positive for LNGFR (CD271) and Thy-1 (CD90), and meet at least one of the following features (a) and (b):
(a) the coefficient of variation for forward scatter in flow cytometry is 40% or less; and
(b) the average size of the cells is 20 μm or less.

23. The method according to claim 20, wherein the human bone marrow-derived high-purity mesenchymal stem cells are a cell population of rapidly proliferating mesenchymal stem cell clones derived from cells positive for LNGFR (CD271) or co-positive for LNGFR (CD271) and Thy-1 (CD90), and meet at least one of the following features (a) and (b):
(a) the coefficient of variation for forward scatter in flow cytometry is 40% or less; and
(b) the average size of the cells is 20 μm or less.

24. The method according to claim 15, wherein the cells are administered at the dose of 1×10⁷ cells per kg body weight of the subject and this administration is repeated weekly four times.

25. The method according to claim 22, wherein the cells are used at a concentration of at least 1×10⁶ cells/ml.

26. The method according to claim 15, wherein the HLA of the donor of the high-purity mesenchymal stem cells does not match the HLA of the subject.

27. The method according to claim 15, wherein the HLA of the donor of the high-purity mesenchymal stem cells matches at least 4 antigens among 6 antigens at three loci in the HLA of the subject.

28. The method according to claim 15, wherein the HLA of the donor of the high-purity mesenchymal stem cells matches at least 3 antigens among 6 antigens at three loci in the HLA of the subject.
